(19) United States
(12) Patent Application Publication
(10) Pub. No.: US 2025/0160373 A1
(43) Pub. Date: May 22, 2025

(54) SOLUBLE FLOUR AND METHODS OF MANUFACTURING SAME

(71) Applicant: CARGILL, INCORPORATED, Wayzata, MN (US)

(72) Inventors: Avi GOLDSTEIN, Golden Valley, MN (US); Xian-Zhong HAN, Maple Grove, MN (US); Varatharajan VAMADEVAN, Plymouth, MN (US)

(21) Appl. No.: 19/027,099

(22) Filed: Jan. 17, 2025

Related U.S. Application Data
(62) Division of application No. 17/254,331, filed on Dec. 21, 2020, now abandoned.
(60) Provisional application No. 62/689,971, filed on Jun. 26, 2018.
(60) Provisional application No. 62/690,649, filed on Jun. 27, 2018.
(60) Provisional application No. 62/767,137, filed on Nov. 14, 2018.

(51) Int. Cl. A23L 7/104; A23L 2/39; A23L 2/52; A23L 7/10; A23L 19/10; A23L 19/15; A23L 27/60; A23L 29/30

(52) U.S. Cl. CPC A23L 7/107; A23L 2/39; A23L 2/52; A23L 7/198; A23L 19/11; A23L 19/15; A23L 27/60; A23L 29/30; A23V 2002/00

(57) ABSTRACT

Described herein is a method of manufacturing a soluble flour comprising preparing a flour-water slurry, adjusting the pH of the slurry to a pH ranging from 3.5-6, adding an enzyme to the pH adjusted slurry in an amount ranging from 0.02-2.0 g enzyme/g flour to form a reaction mixture, cooking the reaction mixture at a temperature ranging from 60-140 C until a dextrose equivalent of 5 to 18 is achieved, inactivating enzyme to obtain a soluble flour; and adjusting pH of the soluble flour to range from 4-5. The soluble flour obtained has a dextrose equivalent value ranging from 5 to 18, a solubility greater than 50% (at 5% solids), and a viscosity between 0.001 and 1.0 Pa*s at temperatures ranging from 20-50 C (at 10% solids).

### CROSS-REFERENCE TO RELATED APPLICATIONS

This application is a Divisional of U.S. patent application Ser. No. 17/254,331, filed Dec. 21, 2020, which is a national phase entry of PCT/US2019/039153, filed Jun. 26, 2019, which claims the benefit of U.S. Provisional Application 62/689,971, filed Jun. 26, 2018, U.S. Patent Application No. 62/690,649, filed Jun. 27, 2018, and U.S. Patent Application No. 62/767,137, filed Nov. 14, 2018, which are hereby incorporated by reference in its entirety.

### TECHNICAL FIELD

This application relates to soluble flour compositions and methods of manufacturing the same.

### BACKGROUND

Consumers are asking for label friendly alternatives to maltodextrin in food and beverage applications. While there is a desire to create label-friendly alternatives, there is also a desire for such alternatives to have similar functionality as that of maltodextrin.

### SUMMARY

Described herein is a method of manufacturing a soluble flour comprising preparing a flour-water slurry, adjusting the pH of the slurry to a pH ranging from 3.5-6.0, adding an enzyme to the pH adjusted slurry in an amount ranging from 0.02-0.1% enzyme relative to weight of the flour to form a reaction mixture, cooking the reaction mixture at a temperature ranging from 60-140 C until a dextrose equivalent of 5 to 18 is achieved, inactivating enzyme to obtain a soluble flour; and adjusting pH of the soluble flour to range from 3-6. The soluble flour obtained has a dextrose equivalent value ranging from 5 to 18, a solubility greater than 50% (at 5% solids), and a viscosity between 0.001 and 1.0 Pa*s at temperatures ranging from 20-50 C (at 10% solids).

### FIGURES

FIG. 1 illustrates the process of manufacturing the soluble flour as described herein.

FIG. 2 graphically illustrates viscosities of the soluble flour as described herein in water (10 wt % soluble flour concentration).

FIG. 3 graphically illustrates the particle size distribution of soluble cassava flour.

FIG. 4 graphically illustrates the particle size distribution of soluble rice flour.

### DETAILED DESCRIPTION

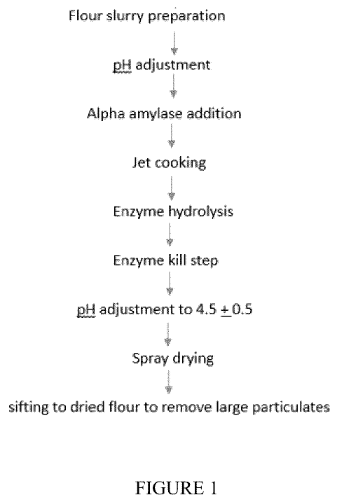

Described herein is a method of manufacturing a soluble flour that can be used in food and beverage applications as a maltodextrin replacement. As used herein, the term “soluble” flour also includes hydrolyzed, enzymatically treated, enzymatically-modified, and/or solubilized flour. Such soluble flour has been treated to promote greater solubility of their principle components in liquids such as water. Further, such soluble flour demonstrates similar functionality as that of maltodextrin, has a desirable “clean flavor”, mouthfeel, and texture suitable for food and beverage applications. An illustration of the general process can be found in FIG. 1. As used herein, the term “soluble” is referencing solubility of flour components in water. As used herein, the term “flour” encompasses (1) non-grain flours and (2) fractionated, non-whole grain flours wherein a portion of bran and germ have been removed.

A first step in the process is the preparing of a slurry made up of flour and water. The flour can be of many sources, for example but not limited to, non-grain sources such as root or tuber sources, and more specifically potato, cassava, sweet potato, taro, yam, arrowroot, lotus root, shoti, Kudzu, banana, waxy cassava, waxy tapioca, or grain flours such as rice, waxy cereal flours, normal cereal flours, or high amylose cereal flours. Sugary-1 mutant flours, and flours containing phytoglycogen can also be used. Flours used as starting materials inherently have low levels of solubility in water.

In preferred aspects, the flour is either cassava flour or rice flour. The slurry comprises about 15 wt % to 30 wt % of the flour, and in more preferred aspects comprises about 20 wt % to 40 wt % flour. In preferred aspects, the slurry is agitated by an agitation means to prevent settling of the flour solids.

The slurry is then pH adjusted to a desirable pH ranging from about 3.5 to 6.0. In preferred aspects from 4.5 to 5.5, in more preferred aspects from 4.7 to 5.3, and in most preferred aspects from 4.8 to 5.2. The pH can be adjusted using acid solutions such as hydrochloric acid.

Once the pH of the slurry is adjusted to fall within the desired range, an enzyme is then added to the slurry. In preferred aspects, the enzyme is an alpha-amylase enzyme, however other bacterial or fungal enzymes may also be used, for example but not limited to iso-, gluco-, beta-, pullulanase, and/or alpha enzymes, and/or combinations thereof. In preferred aspects, the alpha-amylase is a thermal stable alpha-amylase. In preferred aspects, the enzyme is added in an amount ranging from 0.02-2% enzyme relative to weight of the flour, 0.02-0.1% enzyme relative to weight of the flour, and more preferably from 0.045-0.085% enzyme relative to weight of the flour, to form a reaction mixture. The enzyme and slurry make up the reaction mixture. The reaction mixture can be treated at a temperature ranging from 60 C to 140 C, preferably 85 C to 140 C, more preferably 90 C to 100 C, such treatment promotes gelatinization and further solubilization. The reaction mixture is treated until a dextrose equivalent (“DE”) of between 5 and 18 is achieved. In preferred aspects, the cooking would take place until a DE of between 8 and 12 is achieved. Preferably, a jet cooker is used to facilitate the reaction. Once the reaction is complete and the desired DE is achieved, the enzyme is inactivated utilizing common methods such as the addition of acid or heat, and a soluble flour is obtained. The soluble flour is cooled to a temperature ranging from 50 C to 60 C and the pH of the soluble flour is adjusted to a range from about 3 to about 6. The pH can be adjusted using base solutions such as sodium hydroxide. The soluble flour can undergo additional processing, for example spray drying and sifting.

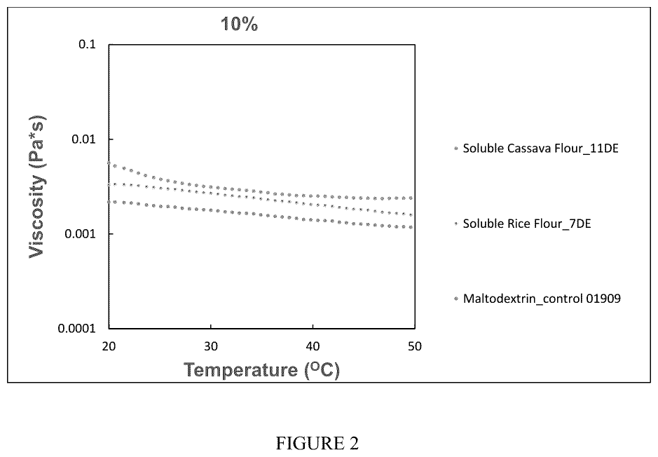

The obtained soluble flour has a solubility ranging from 50% to 100%, (measured at 5% soluble flour concentration also referred to as “5% solids”), and more preferably a solubility ranging from 75% to 85%, and a DE value ranging from 5 to 18 and more preferably a DE value ranging from 8 to 12. The soluble flour also demonstrates desirable viscosity characteristics in water (10 wt % soluble flour concentration also referred to as “10% solids”) ranging from 0.001 and 1 Pa*s. In preferred aspects, the soluble flour has a viscosity ranging from 0.001 and 0.01 Pa*s at temperatures ranging from 20-50 C as shown in FIG. 2. In some aspects, the viscosity characteristics of the soluble flour in water ranges from 0.001 to 0.1 Pa*s.

The soluble flour-water sample was made using an overhead propeller mixer to dissolve soluble solids at 8000 rpm and were tested using an Anton Paar MCR 502 rheometer couette geometry at 20 s⁻¹ of shear rate. The soluble flour also has desirable molecular weight distribution profiles and polydispersity characteristics. Solubility of flours were determined by thoroughly mixing soluble flours in water (5% solids), filtering the sample mixture through filter paper, and determining % Brix of the filtrate using a DR301-95 Digital Refractometer (Kruss GmbH, Hamburg, Germany). In order to determine solubility from the experimentally determined % Brix, one must complete a calculation accounting for the percent of total solids initially added to the system. The DE values of spray dried soluble flours was achieved by quantifying the amount of reducing sugars by Schoolr's method analysis.

In preferred aspects, the soluble flour has a protein content ranging from 0 to 10 wt %, from 0.01 to 10 wt %, and from 0.1 to 10 wt %. In preferred aspects, the soluble flour has a dietary fiber content ranging from 0.5 to 15 wt %.

The soluble flour as described herein is desirable for use in food applications. Notable food applications include but are not limited to beverages, beverage mixes, infant food, medicinal products, food emulsions, convenience foods, bakery, dairy, and snack-based fillings or food products. Beverages and beverage mixes can include instant mixes for hot or cold beverages, flavored milk including chocolate milk, carbonated soft drinks, fruit juices, sports beverages, nutrition beverages, and infant formula. Dairy can include ice cream, yogurt, sour cream, whip cream, and non-dairy vegan alternatives. Convenience foods include but are not limited to salad dressings (pourable and spoonable), sauces (instant and prepare), condiments, puddings, bars, cereals, coatings for cereal, spreads, low-fat spreads, icings, hard candies, soft candies, gummy products, and dry mix seasonings. Bakery can include cookies, cakes, muffins, crackers, pastries, and laminated baked products.

The soluble flour as described herein can be used as at least a partial replacement of maltodextrin in food applications and in many cases can be used as a full replacement of maltodextrin in food applications. Such soluble flower can be an effective maltodextrin replacement in any food application in which maltodextrins are currently used. The soluble flour demonstrates similar functionality (e.g., pH, solubility, and viscosity) as maltodextrin making it a suitable replacement for maltodextrin in food applications. Such replacement allows for consumer-friendly labelling as soluble flours may be more well received by some consumers as compared to maltodextrin.

Further, such soluble flour additionally has the capability to replace maltodextrins in flavor encapsulation applications wherein a flavor emulsion is created and spray dried, to convert a liquid flavor into a solid. In these applications maltodextrins may be used alongside a lipophilic starch, or alternately used alone to create a flavor emulsion. Maltodextrins are typically used in this space due to their ability to form matrices that positively contribute to encapsulation. The soluble flour described herein can replace maltodextrins in this space due to their bland flavor, low viscosity, and low cost. Additionally, soluble flours can replace maltodextrin in plating oil-based flavors.

In preferred aspects, the soluble flour as described herein can be used for instant sauces (e.g., dry mix that is reconstituted to a sauce form by the consumer), prepared sauces, dry mix seasoning, and flavor encapsulation. Such soluble flours can be added in varying amounts and consistently demonstrate similar taste and functionality as maltodextrin.

### EXAMPLES

### Example #1: Manufacturing Process to Prepare Soluble Flour

In a mixing tank, prepare 25% (w/w flour solids) flour slurry in water using 10 Kg flour (wet basis). Table 1 provides information on starting corn maltodextrin, cassava flour, and rice flour materials. Maintain slurry at ambient temperature. Adjust speed of mixing to prevent settling of flour solids.

TABLE 1

| | |
|---|---|
| Maltodextrin | Cargill “Dry MD” 01909 common |
| | corn 10 DE maltodextrin |
| Cassava Flour | Premium Cassava Flour (American Key |
| | Food Products, Closter, New Jersey, USA) |
| Rice Flour | Medium/Short Grain White Rice Flour |
| | Fine-7011 (PGP International, Woodland, |
| | California, USA) |

The pH of the slurry in the tank is adjusted to pH of 4.8-5.2 using 1:1 HCl acid solution. After pH adjustment, continue mixing of the slurry at a gentle speed. Then weigh the required quantity of a thermal-stable alpha amylase enzyme (0.045-0.085% enzyme relative to weight of the flour) and add enzyme into the slurry. After 5 minutes of adding enzyme, check the pH to confirm it is within the range of 4.8-5.2 and record temperature of the slurry once again. Ideal product temperature is between 15-25° C. Continue mixing.

Using water as the feed for the jet cooker, prepare jet cooker and equilibrate the cooking temperature between 110-117° C. and outlet temperature of 95° C. (atmospheric flash in product tank). Once the cooking conditions are set, start feeding the flour slurry into the jet cooker. Record this time as time 0 by starting a stop watch. Keep the stop watch running until the completion of the liquefacts hold and enzyme kill steps to record total liquefaction time. Continue mixing of the slurry while the slurry is being pumped through jet cooker. Keep an eye for the liquefact when it starts to come out of the jet-cooker output. Carefully record this time as the residence time of liquefacts in jet cooker piping. Record this time as “First Liquefaction time”. Collect liquefact into product tank which is equipped with overhead mixer and temperature control up to 95° C.

Hold liquefacts in product tank at 95° C. for a desired holding time that corresponds to a DE (extent of hydrolysis) value desired in final products (typically targeting a DE between 8 and 12). To increase the rate of reaction, additional alpha amylase can be added at this point (0.025-0.035% enzyme relative to weight of the flour). Continue mixing the liquefacts at a slow speed to avoid splashing of hot liquid.

Soon after completion of desired holding times, adjust pH to 2.7-3 at 95° C. and hold for 15 minutes. Continue mixing the liquefacts at a slow speed to avoid splashing of hot liquid. For ensure complete inactivation of enzyme, accurately control temperature and holding time of 15 minutes.

Soon after completion of enzyme kill step, turn off heating by adjusting the steam value and let the slurry cool down to 55-60° C. (use cooling water circulation if needed). Continue mixing of liquefacts at a slow speed to avoid splashing of hot liquid. Adjust pH to 4.5±0.5 in liquefacts using NaOH base solution.

Ensure liquefact prepared by jet cooker is stored in product tank at 50-65° C. while continuously stirring the contents at slow speed to prevent stratification. Transfer approximately 8-10 L of hot liquefact (at 65-75° C.) from product tank to a 5 gallon white plastic pail. Immerse this plastic pail into a 60° C. water bath. Set an overhead mixer to continuously mix the contents in the pail at a slow speed. Record % solids in the liquefact using moisture meter. As needed, adjust % solids in liquefact to lower level using sanitized water to ensure optimum spray drying operation. Feed deionized water into the spray dryer to equilibrate the inlet temperature of the dryer to approximately 200° C. and the outlet temperature to approximately 100° C. Switch the feed from water to liquefaction. Monitor both the inlet and outlet temperatures. Record feed rate, inlet and outlet temperatures of spray dryer. Collect dried product, and store in air tight packaging. Record the final weight and % moisture of the dried hydrolyzed flour product.

Sift dried soluble flour product through a 425 micron (μm) screen to remove any large particulates, or which may have been formed during the drying process.

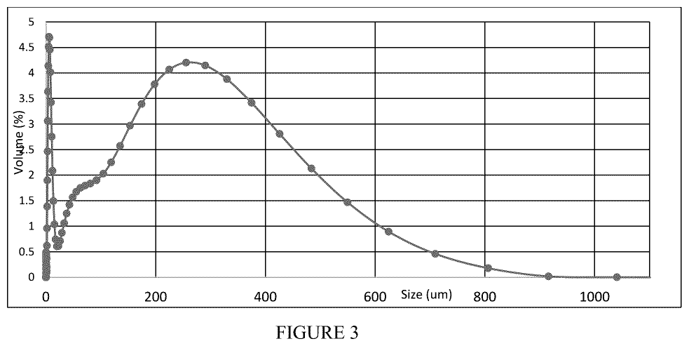

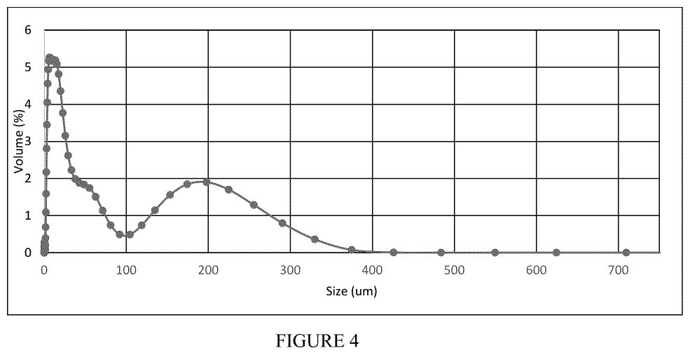

Table 2 provides solubility (measured at 5% solids) and DE data of the soluble flours, Table 3 provides the molecular weight distribution of the soluble fours, and Table 4 provides information on composition per 100 g of final soluble flour product. Note the data in Table 3 represents the mass distribution of the soluble component within the flour products. Molar mass was determined using the SEC MALS RI method described in Example #5. FIGS. 3 and 4 show the particle size distribution of soluble cassava flour and rice flour, respectively.

TABLE 2

| Sample | Solubility (%) | DE |
|---|---|---|
| Cargill Dry MD 01909 | 100 | 10 |
| Soluble Rice Flour | 81 | 8.3 |
| Soluble Cassava Flour | 81 | 11.5 |

TABLE 3

| | Molar Mass | Soluble Cassava | Soluble Rice | Cargill Dry |
|---|---|---|---|---|
| DP | (Da) | Flour | Flour | MD 01909 |
| 1 −> 5 | 0-909 | 0 | 0 | 10 |
| 6 −> 9 | 909-1557 | 0 | 0 | 13.13 |
| 10 −> 19 | 1557-3177 | 0 | 14 | 17.95 |
| 20 −> 45 | 3177-7389 | 0 | 9 | 6.55 |
| 46 −> 125 | 7389-20349 | 8 | 20 | 9.26 |
| 126 −> 280 | 20349-45459 | 65 | 21 | 13.66 |
| 281 −> 600 | 45459-97299 | 16 | 15 | 11.76 |
| 601 −> 1500 | 97299-243828 | 7 | 12 | 11.07 |
| >1500 | >243828 | 3 | 9 | 6.61 |
| | DE | 10 | 8 | 11.5 |
| | Mn (kDa) | 31 | 9 | 2.5 |
| | Mw (kDa) | 59 | 78 | 59 |
| | Polydispersity | 1.9 | 8.6 | 23 |
| | (Mw/Mn) | | | |
| | rh(v)z (nm) | 8 | 9 | <10 |

TABLE 4
Approximate Composition per 100 g final product

| | | | Dietary | | |
|---|---|---|---|---|---|
| | Carbohydrate | Fat | Fiber | Protein | Ash |
| Ingredient | (wt %) | (wt %) | (wt %) | (wt %) | (wt %) |
| Cargill 01909 | 95 | 0.01 | 0 | 0 | 0.01 |
| (common corn | | | | | |
| 10 DE | | | | | |
| maltodextrin) | | | | | |
| Soluble | 81 | <0.5 | 10.8 | 1.5 | 1.26 |
| Cassava Flour- | | | | | |
| Final product | | | | | |
| Soluble | 82 | 0.8 | 0.9 | 6.8 | 0.8 |
| Rice flour- | | | | | |
| final product | | | | | |

### Example #2: Soluble Flours in Chocolate Milk Mix

Soluble cassava flour and soluble rice flour were compared to a control made with 10 DE maltodextrin (prepared as described in Example #1) in chocolate milk mix. The formulation of the chocolate milk mix is provided in Tables 5, 6 and 7.

TABLE 5

| Control Chocolate milk mix | | | | FORMULATION | |
|---|---|---|---|---|---|
| Ingredient Description | Supplier | Product Code | Lot# | % WT | Grams |

| | | | | |
|---|---|---|---|---|
| Whey | Retail | | 38.0270% | 11.4081 |
| Maltodextrin 10 DE | Cargill | 01909 | 30.0000% | 9.0000 |
| Cargill Dry MD* | | | | |
| Cocoa Powder | Cargill | Russet Plus | 30.0000% | 9.0000 |
| Satiaxane CX90 | Cargill | | 0.4410% | 0.1323 |
| Salt | Cargill | Top Flow | 1.0000% | 0.3000 |
| Sucralose | Tate & Lyle | | 0.1500% | 0.0450 |
| Vanilla Flv. (Nesquik | Givaudan | EK-291-228-7 | 0.3820% | 0.1146 |
| vanilla type) | | | | |
| TOTALS | | | 100.00% | 30.00 |

TABLE 6

| Soluble Rice flour Chocolate milk mix | | | | FORMULATION | |
|---|---|---|---|---|---|
| Ingredient Description | Supplier | Product Code | Lot# | % WT | Grams |

| | | | | |
|---|---|---|---|---|
| Whey | Retail | | 38.0270% | 11.4081 |
| Soluble rice flour | Cargill | | 30.0000% | 9.0000 |
| Cocoa Powder | Cargill | Russet Plus | 30.0000% | 9.0000 |
| Satiaxane CX90 | Cargill | | 0.4410% | 0.1323 |
| Salt | Cargill | Top Flow | 1.0000% | 0.3000 |
| Sucralose | Tate & Lyle | | 0.1500% | 0.0450 |
| Vanilla Flv. (Nesquik | Givaudan | EK-291-228-7 | 0.3820% | 0.1146 |
| vanilla type) | | | | |
| TOTALS | | | 100.00% | 30.00 |

TABLE 7

| Soluble Cassava flour Chocolate milk mix | | | | FORMULATION | |
|---|---|---|---|---|---|
| Ingredient Description | Supplier | Product Code | Lot# | % WT | Grams |

| | | | | |
|---|---|---|---|---|
| Whey | Retail | | 38.0270% | 11.4081 |
| Soluble cassava flour | Cargill | | 30.0000% | 9.0000 |
| Cocoa Powder | Cargill | Russet Plus | 30.0000% | 9.0000 |
| Satiaxane CX90 | Cargill | | 0.4410% | 0.1323 |
| Salt | Cargill | Top Flow | 1.0000% | 0.3000 |
| Sucralose | Tate & Lyle | | 0.1500% | 0.0450 |
| Vanilla Flv. (Nesquik | Givaudan | EK-291-228-7 | 0.3820% | 0.1146 |
| vanilla type) | | | | |
| TOTALS | | | 100.00% | 30.00 |

Add listed amounts of whey, cocoa powder, satiaxane CX90, salt, sucralose, and vanilla are to a resealable bag. Shake the bag for up to two minutes. Combine 7.7 grams of the blend to 3.3 grams of maltodextrin or soluble flour (rice or cassava) in a 100 mL tri-pour. Mix well by gently stirring with a spoon. Weigh milk in an appropriate container for heating (8 oz-241.46 g) and heat milk in the microwave for about 20 seconds. Add dry mix to the milk in the warm container. Stir well and evaluate. Table 8 provides evaluation data for the samples. The time for dry mix to go into solution is greater than 2 minutes because an agglomeration step, ordinarily typical for bulking agents in beverage powders, was not carried out. If an agglomeration step is performed, the time will be significantly shorter. Viscosity was determined using a Brookfield Programmable DV-11+ Viscometer (Model: RVRV-II) equipped with LV spindle 61 at a speed of 60 rpm and a temperature of 20° C. *Same amount of soluble cassava and soluble rice was used in both prototypes as the 10 DE control.

TABLE 8

| | | | Time it takes |
|---|---|---|---|
| | | Viscosity | for dry mix to |
| Product | pH | (Cp) | go into solution* |
| Control - 10 DE Maltodextrin | 6.48 | 11.7 | 2:12 |
| Cassava - 2X amylase_savage #1 | 6.46 | 12.8 | 2:38 |
| Rice 2x amylase_Savage #3 | 6.47 | 14.9 | 2:29 |

### Example #3: Soluble Flours in Peach Water Enhancer

Soluble cassava flour and soluble rice flour were compared to a control made with 10 DE maltodextrin (prepared as described in Example #1) in peach water enhancer. The formulation of the peach base is provided in Tables 9, 10 and 11.

TABLE 9

| Peach Water Enhancer Base- Control Formula | | | | FORMULATION | |
|---|---|---|---|---|---|
| Ingredient Description | Supplier | Product Code | Lot# | % WT | Grams |

| | | | | |
|---|---|---|---|---|
| Maltodextrin 10 DE - | Retail | 09109 | 54.997% | 5.736 |
| Cargill Dry MD* | | | | |
| Citric Acid, Anhydrous, Fine | Cargill | | 10.690% | 1.115 |
| Malic Acid | Cargill | | 0.820% | 0.860 |
| ViaTech ™ TS 300+ | Cargill | | 1.439% | 0.150 |
| Granulated Sugar | Cargill | | 29.690% | 3.097 |
| Potassium Citrate | Tate & Lyle | | 0.914% | 0.095 |
| Natural Peach Flavor | Givaudan | | 1.450% | 0.151 |
| TOTALS | | | 100.00% | 10.43 |

TABLE 10

| Peach Water Enhancer Base- Soluble Rice flour | | | | FORMULATION | |
|---|---|---|---|---|---|
| Ingredient Description | Supplier | Product Code | Lot# | % WT | Grams |

| | | | |
|---|---|---|---|
| Soluble rice flour | Retail | 54.997% | 5.736 |
| Citric Acid, Anhydrous, Fine | Cargill | 10.690% | 1.115 |
| Malic Acid | Cargill | 0.820% | 0.860 |
| ViaTech ™ TS 300+ | Cargill | 1.439% | 0.150 |
| Granulated Sugar | Cargill | 29.690% | 3.097 |
| Potassium Citrate | Tate & Lyle | 0.914% | 0.095 |
| Natural Peach Flavor | Givaudan | 1.450% | 0.151 |
| TOTALS | | 100.00% | 10.43 |

TABLE 11

| Peach Water Enhancer Base- Soluble Cassava flour | | | | FORMULATION | |
|---|---|---|---|---|---|
| Ingredient Description | Supplier | Product Code | Lot# | % WT | Grams |

| | | | |
|---|---|---|---|
| Soluble cassava flour | Retail | 54.997% | 5.736 |
| Citric Acid, Anhydrous, Fine | Cargill | 10.690% | 1.115 |
| Malic Acid | Cargill | 0.820% | 0.860 |
| ViaTech ™ TS 300+ | Cargill | 1.439% | 0.150 |
| Granulated Sugar | Cargill | 29.690% | 3.097 |
| Potassium Citrate | Tate & Lyle | 0.914% | 0.095 |
| Natural Peach Flavor | Givaudan | 1.450% | 0.151 |
| TOTALS | | 100.00% | 10.43 |

Add listed amounts of citric acid, malic acid, ViaTech TS 300+, granulated sugar, potassium citrate, and natural peach flavor to a resealable bag. Shake the bag for up to two minutes. Add 4.694 grams of the blend to 5.736 grams of maltodextrin, soluble rice flour, or soluble cassava flour in a 100 mL tri-pour. Mix well by gently stirring with a spoon. Weigh water in an appropriate container and add peach base to the water (453.6 g) Stir well and evaluate. Table 12 provides evaluation data for the samples. Note the time for dry mix to go into solution is greater than 2 minutes because an agglomeration step, ordinarily typical for bulking agents in beverage powders, was not carried out. If an agglomeration step is performed, the time will be significantly shorter.

TABLE 12
Clean Label Maltodextrin Testing Results - Peach Water Enhancer

| | | | Time it takes |
|---|---|---|---|
| | | Viscosity | for peach base to |
| Product | pH | (Cp) | go into solution |
| Control - 10 DE Maltodextrin | 2.66 | 6.4 | 0:25 |
| Cassava - 2X amylase_savage #1 | 2.62 | 8.53 | 1:15 |
| Rice 2x amylase_Savage #3 | 2.57 | 11.7 | 1:10 |

### Example #4: Soluble Flours in Salad Dressing

Soluble cassava flour and soluble rice flour were compared to full fat control dressing and a reduced fat dressing control made with 10 DE maltodextrin (prepared as described in Example #1). The formulations are shown in Tables 13-16.

TABLE 13
Positive Control (Full Fat, 35% Oil)

| INGREDIENT | % | GRAMS |
|---|---|---|
| Water | 49.38 | 246.90 |
| Vegetable Oil | 35.00 | 175.00 |
| Vinegar | 7.00 | 35.00 |
| Sugar | 3.00 | 15.00 |
| Salt | 1.70 | 8.50 |
| Egg Yolk Powder | 1.50 | 7.50 |
| HiForm Starch 12754 | 1.60 | 8.00 |
| Ground Mustard | 0.50 | 2.50 |
| Potassium Sorbate | 0.12 | 0.60 |
| Xanthan gum | 0.20 | 1.00 |
| Total | 100.00 | 500.00 |

TABLE 14
Maltodextrin Control (Reduced Fat, 17.5% Oil)

| INGREDIENT | % | GRAMS |
|---|---|---|
| Water | 48.98 | 244.90 |
| Vegetable Oil | 17.50 | 87.50 |
| Maltodextrin (Cargill | 16.70 | 83.50 |
| Dry MD 01909) | | |
| Vinegar | 7.00 | 35.00 |
| Sugar | 3.00 | 15.00 |
| Salt | 1.70 | 8.50 |
| Egg Yolk Powder | 1.50 | 7.50 |
| HiForm Starch 12754 | 2.80 | 14.00 |
| Ground Mustard | 0.50 | 2.50 |
| Potassium Sorbate | 0.12 | 0.60 |
| Xanthan gum | 0.20 | 1.00 |
| Total | 100.00 | 500.00 |

TABLE 15
Soluble Rice Flour (Reduced Fat, 17.5% Oil)

| INGREDIENT | % | GRAMS |
|---|---|---|
| Water | 48.98 | 244.90 |
| Vegetable Oil | 17.50 | 87.50 |
| Soluble Rice Flour | 16.70 | 83.50 |
| Vinegar | 7.00 | 35.00 |
| Sugar | 3.00 | 15.00 |
| Salt | 1.70 | 8.50 |
| Egg Yolk Powder | 1.50 | 7.50 |
| HiForm Starch 12754 | 2.80 | 14.00 |
| Ground Mustard | 0.50 | 2.50 |
| Potassium Sorbate | 0.12 | 0.60 |
| Xanthan gum | 0.20 | 1.00 |
| Total | 100.00 | 500.00 |

TABLE 16
Soluble Cassava Flour (Reduced Fat, 17.5%
Oil)

| INGREDIENT | % | GRAMS |
|---|---|---|
| Water | 48.98 | 244.90 |
| Vegetable Oil | 17.50 | 87.50 |
| Soluble Cassava Flour | 16.70 | 83.50 |
| Vinegar | 7.00 | 35.00 |
| Sugar | 3.00 | 15.00 |
| Salt | 1.70 | 8.50 |
| Egg Yolk Powder | 1.50 | 7.50 |
| HiForm Starch 12754 | 2.80 | 14.00 |
| Ground Mustard | 0.50 | 2.50 |
| Potassium Sorbate | 0.12 | 0.60 |
| Xanthan gum | 0.20 | 1.00 |
| Total | 100.00 | 500.00 |

Water is combined with the dry ingredients and mixed in a Cuisinart food processor for approximately 3 minutes. The oil is slowly added to the mixer and mixed for approximately 5 minutes. Vinegar is then added and mixed for approximately 1 minutes. Bostwick measurements are then measured and described in Table 17. Measurements were completed utilizing Bostwick Consistometer (CSC Scientific Company, Inc., Fairfax, Virginia, USA) with a sample loading weight of 100 grams, and a measurement window of 30 seconds. Samples were also tested using an Anton Paar MCR 502 rheometer couette geometry at shear rates of 0.1 to 100 s⁻¹ of at a controlled temperature of 4° C. and described in Table 18.

TABLE 17

| | Initial Bostwick: | | 24 hours Bostwick: | |
|---|---|---|---|---|
| | Sample temperature | | Sample temperature | |
| Sample | 20° C. (30 sec) | | 4° C. (30 sec) | |
| Full Fat Control | 7.5 | cm | 7 | cm |
| (35% Oil) | | | | |
| Maltodextrin Control | 16 | cm | 14 | cm |
| (Cargill Dry MD | | | | |
| 01909) (17.5% Oil) | | | | |
| Soluble Rice Flour | 10 | cm | 9.5 | cm |
| Soluble Cassava Flour | 6 | cm | 5 | cm |

TABLE 18

| Formula | Viscosity (Pa*s) |
|---|---|
| Salad Dressing- Full Fat Control | 16.6 |
| Salad Dressing- Control Maltodextrin | 8.67 |
| Salad Dressing- Soluble Cassava Flour | 55 |
| Salada Dressing-Soluble Rice Flour | 23.5 |

### Example #5: Molar Mass Distribution Method

### Instruments:

HPLC: Agilent 1260 Infinity System
Multi-angle light scattering Detector (MALS): Wyatt Technology DAWN HELEOS II
Refractive Index Detector (RI): Wyatt Technology Optilab TrEX
Column Heater
Instrument Set Up: HPLC-Column heater-MALS-RI

### Columns:

Phenomenex Phenogel 10 u (7.8×300 mm)
   Columns in series: guard column-10E6A-10E5A-10E3A
Column Temperature: 55° C.

### Sample Preparation Procedure:

1. Add 100 mg sample into a 25×150 mm culture tube with cap.
   Note: Before sample addition remove particles from tube & cap using canned air (Duster)
2. Add 20 mL of 50 mM LiBr 100% DMSO mobile phase (isocractic, run time; 70 minutes) to the tube using a 25 mL graduated cylinder.
   Note: Make sure to wash down any sample stuck on the tube sides.
3. Add mini-stir bar and immediately set on stir plate.
4. Stir samples for 1 hour at low rpm.
5. Place tubes in water bath.
6. Heat water until vigorous boil with continuous sample solution stirring.
7. Turn off hot plate.
8. Leave samples in the water bath stirring on hot plate until tubes are at room temperature.
9. De-pressurize the tubes by quickly loosening then re-tightening the cap.
10. Mix samples with vortex mixer.
11. Place samples on stir plate and stir overnight.
12. Filter sample through a 1 um PTFE syringe filter into a 2 mL HPLC vial.
13. Analyze samples by SEC-MALLS-RI system.

1. A method of manufacturing a soluble flour comprising:
i. preparing a flour-water slurry;
ii. adjusting the pH of the slurry to a pH ranging from 3.5-6.0;
iii. adding an enzyme to the pH adjusted slurry in an amount ranging from 0.02-2% enzyme relative to weight of the flour to form a reaction mixture;
iv. cooking the reaction mixture at a temperature ranging from 60-140° C. until a dextrose equivalent of 5 to 18 is achieved;
V. inactivating the enzyme to obtain an initial soluble flour; and
vi. adjusting the pH of the initial soluble flour to range from 3-6 to form the soluble flour.

2. The method of claim 1, wherein step (ii) comprises adjusting the pH of the slurry to a pH ranging from 4.8-5.2.

3. The method of claim 1, wherein the enzyme is added in an amount ranging from 0.045-0.085% enzyme relative to weight of the flour.

4. The method of claim 1, wherein the flour-water slurry comprises 20-40 wt % flour.

5. The method of claim 1, wherein in step (iv) the reaction mixture cooking takes place in a jet cooker.

6. The method of claim 1, wherein the temperature during cooking ranges from 90 C to 100 C.

7. The method of claim 1, further comprising spray drying the soluble flour following step (vi).

8. The method of claim 1, wherein the flour in the flour-water slurry is a non-grain flour.

9. The method of claim 8, wherein the non-grain flour is a root or tuber flour.

10. The method of claim 8, wherein the non-grain flour is potato, cassava, sweet potato, taro, yam, arrowroot, lotus root, shoti, Kudzu, banana, waxy cassava, or waxy tapioca flour.

11. The method of claim 1, wherein the flour in the four-water slurry is rice, waxy cereal, normal cereal, or high amylose cereal flours.

12. The method of claim 1, wherein the enzyme is an alpha-amylase.

13. The method of claim 12, wherein the alpha-amylase is a thermal stable alpha-amylase.

14. The method of claim 1, wherein the soluble flour has a dextrose equivalent ranging from 8 to 12.

15. The method of claim 1, wherein the soluble flour has a solubility greater than 50% at 5% solids.

16. The method of claim 1, wherein the soluble flour has a solubility ranging from 50% to 100% at 5% solids.

17. The method of claim 1, wherein the soluble flour has a viscosity between 0.001 and 1.0 Pa*s at temperatures ranging from 20-50° C. at 10% solids.

18. The method of claim 1, wherein the soluble flour has a solubility greater than 50% and up to 85% at 5% solids.

19. The method of claim 1, wherein the soluble flour comprises 0.1 wt % to 10 wt % protein.

20. The method of claim 1, wherein the soluble flour comprises 0.5 wt % to 15 wt % dietary fiber.
